(19) United States
(12) Patent Application Publication
(10) Pub. No.: US 2018/0184886 A1
(43) Pub. Date: Jul. 5, 2018

(54) ENDOSCOPE SHEATH AND ENDOSCOPE SYSTEM

(71) Applicant: OLYMPUS CORPORATION, Tokyo (JP)

(72) Inventors: Naoya SUGIMOTO, Tokyo (JP); Yoshiro OKAZAKI, Tokyo (JP); Hiroki HIBINO, Tokyo (JP); Kazutoshi KUMAGAI, Tokyo (JP); Masayuki KOBAYASHI, Tokyo (JP)

(73) Assignee: OLYMPUS CORPORATION, Tokyo (JP)

(21) Appl. No.: 15/907,790

(22) Filed: Feb. 28, 2018

Related U.S. Application Data
(63) Continuation of application No. PCT/JP2015/083100, filed on Nov. 25, 2015.

(51) Int. Cl. A61B 1/00; A61B 1/01

(52) U.S. Cl. CPC A61B 1/00135; A61B 1/018; A61B 1/01; A61B 1/00142

(57) ABSTRACT

Provided is an endoscope sheath including an elongated body having flexibility, the body having a first lumen through which an endoscope passes in a longitudinal direction of the body and a second lumen through which a guide wire passes in the longitudinal direction. A direction in which the second lumen opens at the distal end of the body gradually diverges from a direction in which the first lumen opens at the distal end, in a forward direction from the distal end.

This is a continuation of International Application PCT/JP2015/083100, with an international filing date of Nov. 25, 2015, which is hereby incorporated by reference herein in its entirety.

### TECHNICAL FIELD

The present invention relates to endoscope sheaths and endoscope systems.

### BACKGROUND ART

There is a known guide-wire introduction device including a first lumen through which a guide wire can pass, and a second lumen through which an endoscope can pass (for example, see Patent Literature 1). In this guide-wire introduction device, whether the guide wire has been introduced into the pericardium can be directly checked with the endoscope by making the guide wire introduced through the first lumen pass through the field of view of the endoscope inserted through the second lumen.

### CITATION LIST

### Patent Literature

{PTL 1} Japanese Unexamined Patent Application Publication No. 2014-18299

### SUMMARY OF INVENTION

An aspect of the present invention is directed to an endoscope sheath including an elongated body having flexibility, the body including a first lumen through which an endoscope passes in a longitudinal direction of the body and a second lumen through which a guide wire passes in the longitudinal direction of the body. A direction in which the second lumen opens at the distal end of the body gradually diverges from a direction in which the first lumen opens at the distal end, in a forward direction from the distal end.

Another aspect of the present invention is directed to an endoscope system including: any one of the above-described endoscope sheaths; an endoscope that is inserted through the first lumen of the endoscope sheath; and a guide wire that is inserted through the second lumen of the endoscope sheath.

### BRIEF DESCRIPTION OF DRAWINGS

FIG. 1 is a longitudinal cross section showing an endoscope system according to an embodiment of the present invention.

FIG. 2 is a side view showing the endoscope system in FIG. 1.

FIG. 3 is a lateral cross section of an endoscope sheath according to the embodiment of the present invention, the endoscope sheath being provided in the endoscope system in FIG. 1.

FIG. 4 is a partial longitudinal cross section of the endoscope system in FIG. 1, taken along a second lumen.

FIG. 5 is a schematic view showing a state in which the distal end portion of the endoscope sheath of the endoscope system in FIG. 1 is inserted into the pericardial cavity.

FIG. 6 is a schematic view showing a state in which, from the state shown in FIG. 5, a guide wire is projected to ensure a space inside the pericardial cavity.

FIG. 7 is an exploded longitudinal cross section showing a modification of the endoscope sheath in FIG. 3.

FIG. 8 is a lateral cross section of a separator provided in the endoscope sheath in FIG. 7.

FIG. 9 is a side view of the assembled endoscope sheath in FIG. 7.

FIG. 10 is a lateral cross section showing a modification of the separator in FIG. 8.

### DESCRIPTION OF EMBODIMENTS

An endoscope system 1 and an endoscope sheath 2 according to an embodiment of the present invention will be described below with reference to the drawings.

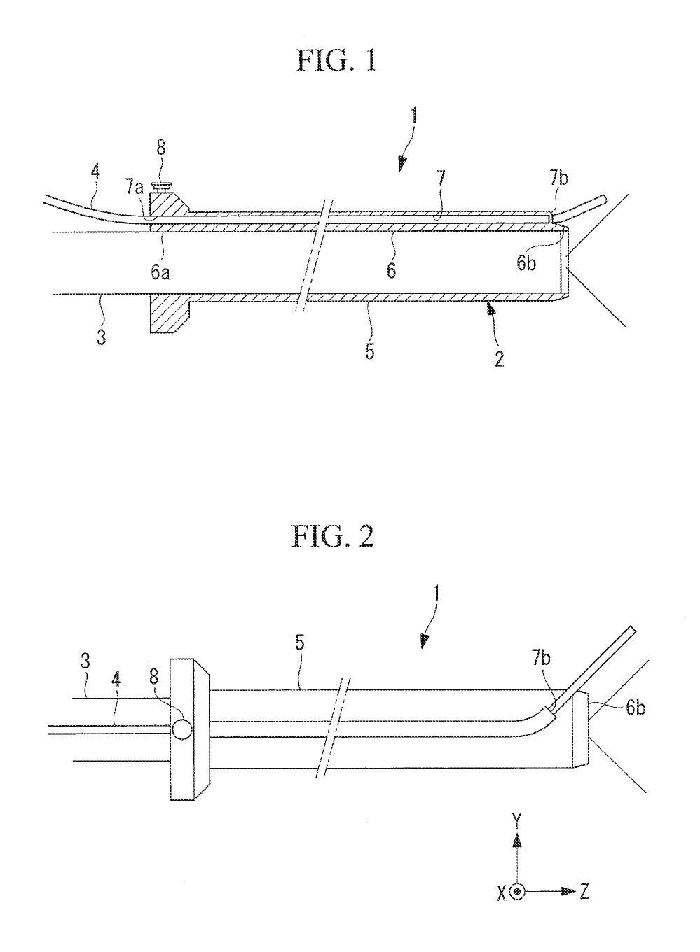

As shown in FIGS. 1 and 2, the endoscope system 1 according to this embodiment includes an endoscope sheath 2 according to this embodiment, an endoscope 3, and a guide wire 4.

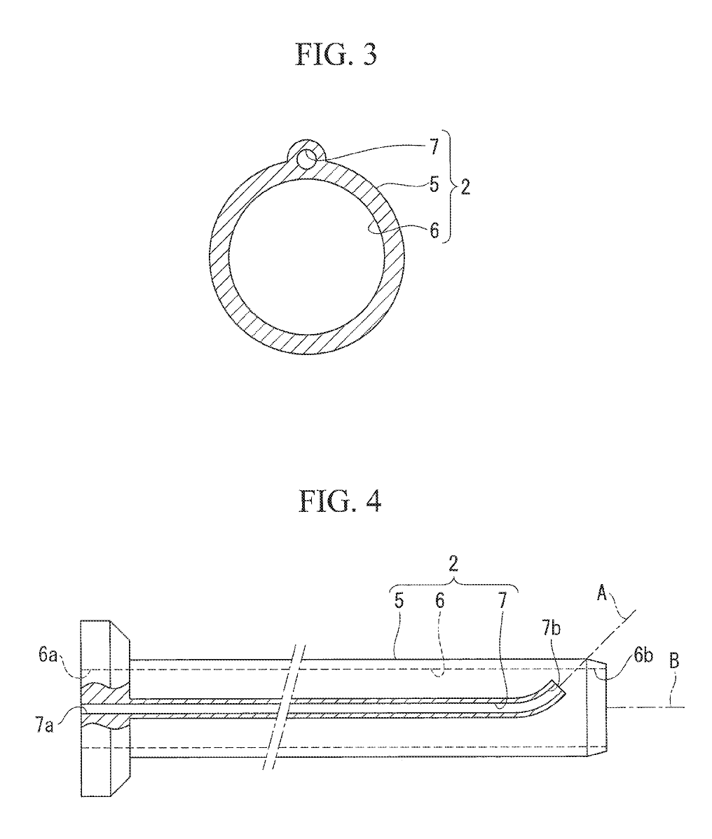

As shown in FIGS. 1 and 3, the endoscope sheath 2 according to this embodiment includes an elongated, flexible tubular sheath body (body) 5 and one first lumen 6 and one second lumen 7 passing therethrough in the longitudinal direction. The first lumen 6 has a diameter allowing the endoscope 3 to pass therethrough and opens in the distal end face and the base end face of the sheath body 5. The first lumen 6 is configured to allow the endoscope 3 to observe a subject located in front of the distal end face by exposing the endoscope 3, which is inserted from an opening 6a in the base end face and is disposed so as to penetrate in the longitudinal direction, from the opening 6b in the distal end face.

The second lumen 7 has a diameter allowing the guide wire 4 to pass therethrough and opens near the distal end and the base end face of the sheath body 5. The second lumen 7 allows the distal end portion of the guide wire 4, which is inserted from the opening 7a in the base end face and is disposed so as to penetrate in the longitudinal direction, to project from the opening 7b at the distal end.

As shown in FIG. 4, in this embodiment, the second lumen 7 is disposed parallel to the first lumen 6 over substantially the entire length from the base end face of the sheath body 5 and is curved near the distal end of the sheath body 5. In the example shown in FIG. 4, the direction in which the second lumen 7 is curved is a direction parallel to a tangent plane tangential to the contour of the first lumen 6, the contour being located between the first lumen 6 and the second lumen 7.

Hence, the direction in which the opening 7b of the second lumen 7 opens at the distal end of the sheath body 5 (i.e., the direction of the center line A) gradually diverges from the direction in which the opening 6b of the first lumen 6 opens at the distal end face of the sheath body 5 (i.e., the direction of the center line B).

Furthermore, a connector 8 for connecting a water/gas supply/discharge pipe (not shown), which supplies gas or liquid to the inside of the body through the sheath body 5, is disposed at the base end portion of the sheath body 5. For example, as shown in FIG. 2, the connector 8 is disposed at a position (X direction) substantially 90° away, in the circumferential direction, from the direction (Y direction) in which the opening 7b at the distal end of the second lumen 7 opens.

The operation of the thus-configured endoscope system 1 according to this embodiment will be described below.

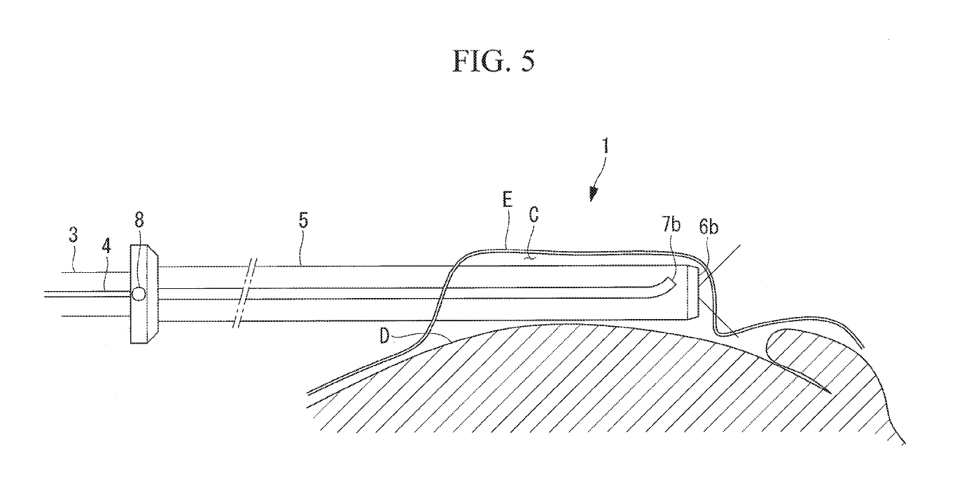

When the inside of the pericardial cavity C is to be observed and treated with the endoscope system 1 according to this embodiment, the sheath body 5 is inserted through a hole extending from below the ensiform cartilage to the inside of the pericardial cavity C. As shown in FIG. 5, the base end face of the sheath body 5 is disposed outside the body, and the distal end face of the sheath body 5 is disposed inside the pericardial cavity C.

In this state, the guide wire 4 inserted from the opening 7a at the base end of the second lumen 7 is projected from the opening 7b at the distal end of the second lumen 7 into the pericardial cavity C. The guide wire 4 is introduced so as to conform to the shape of the second lumen 7, is curved along the curve of the distal end portion of the second lumen 7, and is projected into the pericardial cavity C.

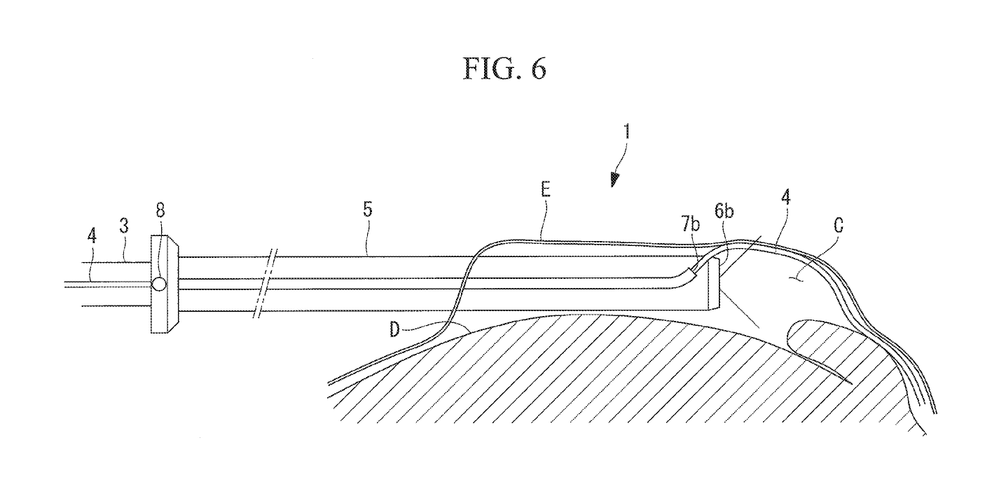

Since the opening 7b at the distal end of the second lumen 7 opens in the direction in which the center line A thereof diverges from the center line B of the first lumen 6, the guide wire 4 projecting from the opening 7a at the distal end of the second lumen 7 is projected so as not to extend across in front of the opening 6b of the first lumen 6, as shown in FIG. 6. As a result, because the guide wire 4 does not block the view of the endoscope 3 introduced through the first lumen 6, observation with the endoscope 3 and treatment with a treatment instrument (not shown) introduced through the endoscope 3 are not inhibited, which is advantageous.

Furthermore, in the endoscope sheath 2 and the endoscope system 1 according to this embodiment, because the guide wire 4 projecting from the second lumen 7 projects in a direction away from the first lumen 6, by adjusting the angle of the endoscope sheath 2 about the longitudinal axis, the guide wire 4 can be projected in a direction away from the surface of the heart D, as shown in FIG. 6. This leads to an advantage in that it is possible to push, with the guide wire 4, the pericardium E in a direction away from the surface of the heart D and thus to ensure, inside the pericardial cavity C, the wide space needed for observation and treatment.

In particular, since the guide wire 4 projecting from the second lumen 7 projects in an oblique direction from the opening 7b provided near the distal end face of the sheath body 5, the guide wire 4 comes into contact with the pericardium E at a position near the position where it is supported by the second lumen 7 and thus presses the pericardium E with a relatively large force. Hence, it is possible to more reliably push and expand the space for observation. In particular, the pericardium E is pushed and expanded on the opposite side from the heart D side, on which the observation or treatment is performed, so that observation or treatment can be easily performed.

Furthermore, in this embodiment, since the connector 8 for connecting the water/gas supply/discharge pipe is disposed at a position substantially 90° away, in the circumferential direction, from the direction in which the guide wire 4 projects from the second lumen 7, it is possible to dispose the pipe connected to the connector 8 to inflate the inside of the body cavity at a position where it does not obstruct the operator operating the endoscope 3.

Specifically, when the guide wire 4 is inserted through the second lumen 7 in the sheath body 5 introduced into the pericardial cavity C from below the ensiform cartilage of a patient who is lying on their back, the heart D is below and the pericardium E is above the guide wire 4. Hence, it is desirable that the opening 7b of the second lumen 7 at the distal end of the sheath body 5 be disposed so as to face upward. Then, by disposing the connector 8 at a position substantially 90° away, in the circumferential direction, from the direction in which the guide wire 4 projects from the second lumen 7, it is possible to dispose the connector 8 not on the operator side, but on the side opposite from the operator. Thus, the pipe connected to the connector 8 is prevented from obstructing the manipulation by the operator.

Note that the connector 8 may be positioned in the same direction as the direction in which the opening 7b of the second lumen 7 opens, besides the position directly opposite from the operator. Thus, the connector 8 may be disposed within the range of 0° to 90° from the direction in which the opening 7b of the second lumen 7 opens.

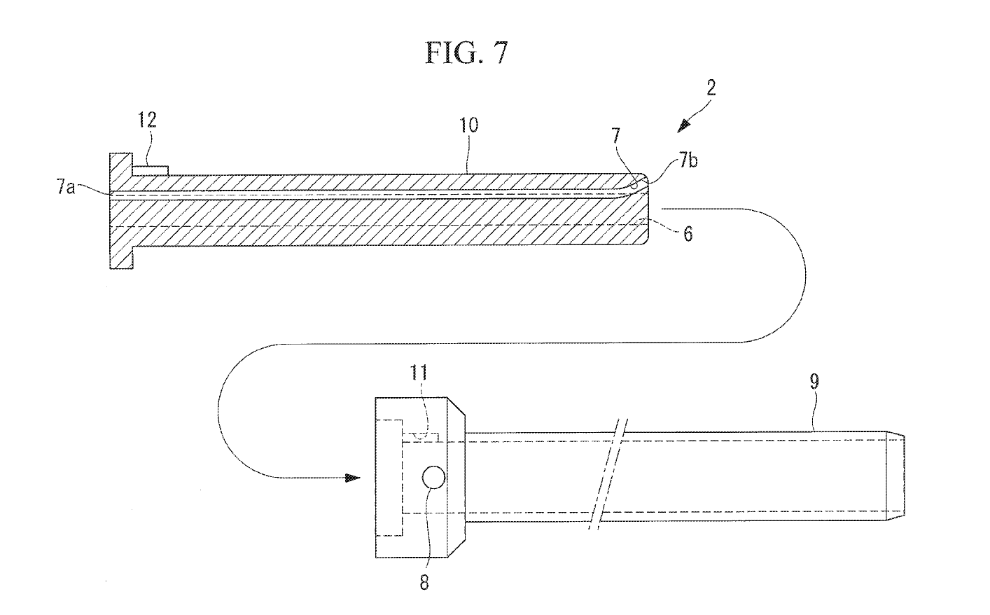

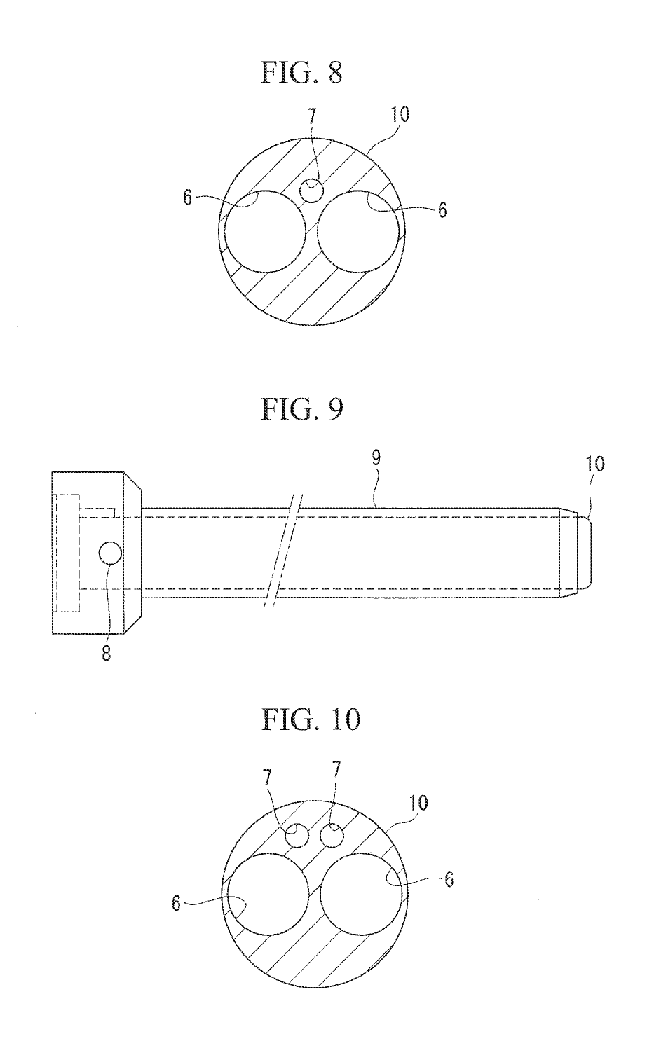

Furthermore, in this embodiment, although the first lumen 6 and the second lumen 7 are provided in the single sheath body 5, instead, as shown in FIGS. 7 to 9, the endoscope sheath 2 may have a cylindrical sheath body 9 and a separator 10 fitted inside the sheath body 9, and, as shown in FIGS. 7 and 8, the first lumen 6 and the second lumen 7 may be provided in the separator 10. In this case, positioning portions, such as a keyway 11 in the inner surface of the sheath body 9 and a key 12 on the outer surface of the separator 10, may be provided to position the sheath body 9 and the separator 10 relative to each other in the circumferential direction.

Furthermore, as for the first lumen 6, two or more may be provided in the sheath body 9 or the separator 10, as shown in FIG. 8. In this case, a space for providing the second lumen 7 may be provided between the first lumens 6. Accordingly, as shown in FIG. 7, the second lumen 7 may be configured to bend radially outward in the radial direction of the sheath body 5 or the separator 10.

Furthermore, as for the second lumen 7, two or more may also be provided, as shown in FIG. 10.

### REFERENCE SIGNS LIST

As a result, the above-described embodiment leads to the following aspects.

An aspect of the present invention is directed to an endoscope sheath including an elongated body having flexibility, the body including a first lumen through which an endoscope passes in a longitudinal direction of the body and a second lumen through which a guide wire passes in the longitudinal direction of the body. A direction in which the second lumen opens at the distal end of the body gradually diverges from a direction in which the first lumen opens at the distal end, in a forward direction from the distal end.

According to this aspect, the endoscope is introduced into a living body through the first lumen of the body disposed in the living body, and the guide wire is introduced into the living body through the second lumen. The guide wire is projected in a direction away from the first lumen when projected from the opening of the second lumen at the distal end of the body. Hence, the field of view of the endoscope introduced through the first lumen is not blocked by the guide wire, making it possible to improve the ease of manipulation with the endoscope or a treatment instrument inserted through the endoscope.

In the above-described aspect, an opening of the second lumen at the distal end of the body may be provided in a vicinity of the outer circumference of the body.

With this configuration, the guide wire inserted through the second lumen comes into contact with and presses the surrounding living body tissue immediately after projecting from the opening of the second lumen at the distal end of the body. By pressing the living body tissue with a portion that is supported by the second lumen and thus can easily exert a large pressing force, a space can be more reliably ensured in the living body.

In the above-described aspect, the second lumen may be divided in a vicinity of the distal end of the body.

With this configuration, by twisting the guide wire about the axis thereof in the second lumen, the guide wire can be advanced in any of the directions in which the second lumen is divided. No matter in what direction the guide wire is projected, it does not block the view of the endoscope. Furthermore, it is possible to select the direction in which the guide wire is projected according to the direction in which the living body tissue to be pressed by the guide wire adjoins the body.

Furthermore, in the above-described aspect, the body may include, at a base end portion thereof, a connector for connecting a water/gas supply/discharge pipe, and the opening of the second lumen at the distal end of the body may be arranged at a position substantially 90° away from the connector in a circumferential direction.

When an operator inserts an endoscope sheath into the pericardial cavity from below the ensiform cartilage of a patient who is lying on their back and performs manipulation while pressing and expanding the pericardium upward with the guide wire inserted through the second lumen, it is desirable that the pipe connected to the connector be disposed on the side opposite from the operator because it obstructs the manipulation by the operator if disposed on the operator's side.

With this configuration, it is possible to dispose the pipe connected to the connector at a position where it does not obstruct the operator and to spread out the pericardium to ensure a space inside the pericardial cavity.

Furthermore, in the above-described aspect, the endoscope sheath may further include a separator fitted into the body, the separator including the first lumen and the second lumen, wherein a direction in which the second lumen opens at the distal end of the separator gradually diverges from the direction in which the first lumen opens at the distal end, in the forward direction from the distal end.

With this configuration, the separator is fitted into the body disposed in the living body, and the endoscope is introduced into the living body through the first lumen of the separator, and the guide wire is introduced into the living body through the second lumen. The guide wire is projected in a direction away from the first lumen when projected from the opening of the second lumen at the distal end of the separator. Hence, the field of view of the endoscope introduced through the first lumen is not blocked by the guide wire, making it possible to improve the ease of manipulation with the endoscope or a treatment instrument introduced through the endoscope.

Furthermore, in the above-described aspect, the body may be, at a base end portion thereof, provided with a connector for connecting a water/gas supply/discharge pipe, and the endoscope sheath may include a positioning portion that is configured to position the body and the separator in a circumferential direction such that an opening of the second lumen at the distal end of the separator is provided at a position substantially 90° away from the connector in the circumferential direction.

With this configuration, it is possible to dispose the pipe connected to the connector at a position where it does not obstruct the operator and to spread out the pericardium to ensure a space inside the pericardial cavity.

Another aspect of the present invention is directed to an endoscope system including: any one of the above-described endoscope sheaths; an endoscope that is inserted through the first lumen of the endoscope sheath; and a guide wire that is inserted through the second lumen of the endoscope sheath.

The present invention has an advantage in that the introduced guide wire ensures a working space for the endoscope, making it possible to improve the ease of manipulation with the endoscope or a treatment instrument introduced through the endoscope.
1 endoscope system
2 endoscope sheath
3 endoscope
4 guide wire
5 body
6 first lumen
7 second lumen
7b opening
8 connector
9 sheath body
10 separator
11 keyway (positioning portion)
12 key

1. An endoscope sheath comprising an elongated body having flexibility, the body including a first lumen through which an endoscope passes in a longitudinal direction of the body and a second lumen through which a guide wire passes in the longitudinal direction of the body,
wherein a direction in which the second lumen opens at the distal end of the body gradually diverges from a direction in which the first lumen opens at the distal end, in a forward direction from the distal end.

2. The endoscope sheath according to claim 1, wherein an opening of the second lumen at the distal end of the body is provided in a vicinity of an outer circumference of the body.

3. The endoscope sheath according to claim 1, wherein the second lumen is divided in a vicinity of the distal end of the body.

4. The endoscope sheath according to claim 1, wherein
the body comprises, at a base end portion thereof, a connector for connecting a water/gas supply/discharge pipe, and
the opening of the second lumen at the distal end of the body is arranged at a position substantially 90° away from the connector in a circumferential direction.

5. The endoscope sheath according to claim 1, further comprising a separator fitted into the body, the separator including the first lumen and the second lumen,
wherein a direction in which the second lumen opens at a distal end of the separator gradually diverges from the direction in which the first lumen opens at the distal end, in the forward direction from the distal end.

6. The endoscope sheath according to claim 5, wherein
the body is, at a base end portion thereof, provided with a connector for connecting a water/gas supply/discharge pipe, and
the endoscope sheath includes a positioning portion that is configured to position the body and the separator in a circumferential direction such that an opening of the second lumen at the distal end of the separator is provided at a position substantially 90° away from the connector in the circumferential direction.

7. An endoscope system comprising:
the endoscope sheath according to claim 1;
an endoscope that is inserted through the first lumen of the endoscope sheath; and
a guide wire that is inserted through the second lumen of the endoscope sheath.

8. The endoscope sheath according to claim 2, wherein
the body comprises, at a base end portion thereof, a connector for connecting a water/gas supply/discharge pipe, and
the opening of the second lumen at the distal end of the body is arranged at a position substantially 90° away from the connector in a circumferential direction.

9. An endoscope system comprising:
the endoscope sheath according to claim 2;
an endoscope that is inserted through the first lumen of the endoscope sheath; and
a guide wire that is inserted through the second lumen of the endoscope sheath.
